(19) United States
(12) Patent Application Publication
(10) Pub. No.: US 2024/0188889 A1
(43) Pub. Date: Jun. 13, 2024

(54) FLASH LED AND HEART RATE MONITOR LED INTEGRATION AND RELATED DEVICES AND METHODS

(71) Applicant: META PLATFORMS TECHNOLOGIES, LLC, Menlo Park, CA (US)

(72) Inventors: Szymon Michal Tankiewicz, San Jose, CA (US); Yizhi Xiong, Foster City, CA (US)

(21) Appl. No.: 17/515,583

(22) Filed: Nov. 1, 2021

Related U.S. Application Data
(60) Provisional application No. 63/119,243, filed on Nov. 30, 2020.

(51) Int. Cl. A61B 5/00; A61B 5/024

(52) U.S. Cl. CPC A61B 5/681; A61B 5/0077; A61B 5/024; A61B 5/742

(57) ABSTRACT

The disclosed watch may include a watch band, a watch body that includes a front surface and a rear surface, at least one light emitting diode, and an image sensor. The watch body may be configured to contact a user's wrist when the watch body is attached to the watch band and donned by the user. The at least one light emitting diode and the image sensor may be on the rear surface of the watch body. The at least one light emitting diode may be configured to provide a light source for a heart rate monitoring function of the watch when the watch body is attached to the watch band and to provide a light source for the image sensor when the watch body is detached from the watch band. Various other related methods and systems are also disclosed.

### CROSS-REFERENCE TO RELATED APPLICATION

This application claims the benefit of U.S. Provisional Application No. 63/119,243, filed 30 Nov. 2020, the entire disclosure of which is incorporated herein by reference.

### BRIEF DESCRIPTION OF THE DRAWINGS

The accompanying drawings illustrate a number of example embodiments and are a part of the specification. Together with the following description, these drawings demonstrate and explain various principles of the present disclosure.

FIG. 1A is a plan view of an example wristband system, according to at least one embodiment of the present disclosure.

FIG. 1B is a side view of the example wristband system of FIG. 1A, with a watch body thereof decoupled from a wristband thereof, according to at least one embodiment of the present disclosure.

FIG. 2 is a perspective view of an example wristband system, according to at least one embodiment of the present disclosure.

FIG. 3 is a perspective view of an integrated flash LED and heart rate monitor LED, according to at least one embodiment of the present disclosure.

FIG. 4A is a plan view of an example watch body with an integrated flash LED and heart rate monitor LED, according to at least one embodiment of the present disclosure.

FIG. 4B is a plan view of another example watch body with a flash LED and a heart rate monitor LED, according to at least one embodiment of the present disclosure.

FIGS. 5A and 5B illustrate shadows introduced in the capture of an image.

FIGS. 6A and 6B are diagrams illustrating a field of view of a flash LED.

FIG. 7 is an illustration of example augmented-reality glasses that may be used in connection with embodiments of this disclosure.

FIG. 8 is an illustration of an example virtual-reality headset that may be used in connection with embodiments of this disclosure.

FIG. 9 is an illustration of example haptic devices that may be used in connection with embodiments of this disclosure.

FIG. 10 is an illustration of an example virtual-reality environment according to embodiments of this disclosure.

FIG. 11 is an illustration of an example augmented-reality environment according to embodiments of this disclosure.

FIGS. 12A and 12B are illustrations of an example human-machine interface configured to be worn around a user's lower arm or wrist.

FIGS. 13A and 13B are illustrations of an example schematic diagram illustrating internal components of a wearable system.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the present disclosure covers all modifications, equivalents, and alternatives falling within the scope of the appended claims.

### DETAILED DESCRIPTION OF EXAMPLE EMBODIMENTS

Wearable devices may be configured to be worn on a user's body part, such as a user's wrist or arm. Such wearable devices may be configured to perform various functions. A wristband system may be an electronic device worn on a user's wrist that performs functions such as monitoring heart rate functions, capturing images, delivering content to the user, executing social media applications, executing artificial-reality applications, messaging, web browsing, sensing ambient conditions, interfacing with head-mounted displays, monitoring the health status associated with the user, etc.

The present disclosure is directed to a watch body that includes a light emitting diode (LED) that may function as a camera flash. In some examples, the camera flash LED and a heart rate monitor (HRM) LED may be integrated into a single package. The single package may include a multichip module on an interconnection substrate. The single package may be mounted on a printed circuit board (PCB) within a watch body (e.g., a wrist-worn smartwatch). The single package may also integrate the image sensor and/or an analog front end for processing analog signals. In some examples, the camera flash LED and the HRM LED may be mounted individually on a printed circuit board disposed within the watch body. In additional examples, the HRM LED may be operable as a camera flash. By operating the HRM LED as a camera flash, better image exposure may be obtained without the need for an additional component (e.g., a separate flash LED). Integration of the camera flash LED and the HRM LED may reduce package size, cost, and/or power consumption. The operation of the integrated LED may switch between camera flash operation and HRM operation based on data from a sensor that monitors whether the watch body is worn against the user's wrist, attached to a watch band, and/or detached from the watch band.

The watch body may include a coupling mechanism for electrically and mechanically coupling (e.g., attaching) the watch body to the watch band. The wristband system may have a split architecture that allows the watch band and the watch body to operate both independently and in communication with one another. The mechanical architecture may include a coupling mechanism on the watch band and/or the watch body that allows a user to conveniently attach and detach the watch body from the watch band as desired. In some embodiments, the LED may be configured to provide a light source for the HRM sensor when the watch body is attached to a watch band and the LED may be configured to provide a light source for the image sensor when the watch body is not attached to the watch band and/or not worn against the user's wrist.

The wristband system may be used in conjunction with an artificial-reality (AR) system. Sensors of the wristband system (e.g., HRM sensors, image sensors, inertial measurement unit, etc.) may be used to enhance an AR application running on the AR system. Further, the watch band may include sensors that measure biometrics of the user. For example, the watch band may include HRM sensors disposed on an inside surface of the watch band that monitor heart rate functions of the user. Signals sensed by the HRM sensor may be processed and used to provide a user with an enhanced interaction with a physical object and/or a virtual object in an AR environment.

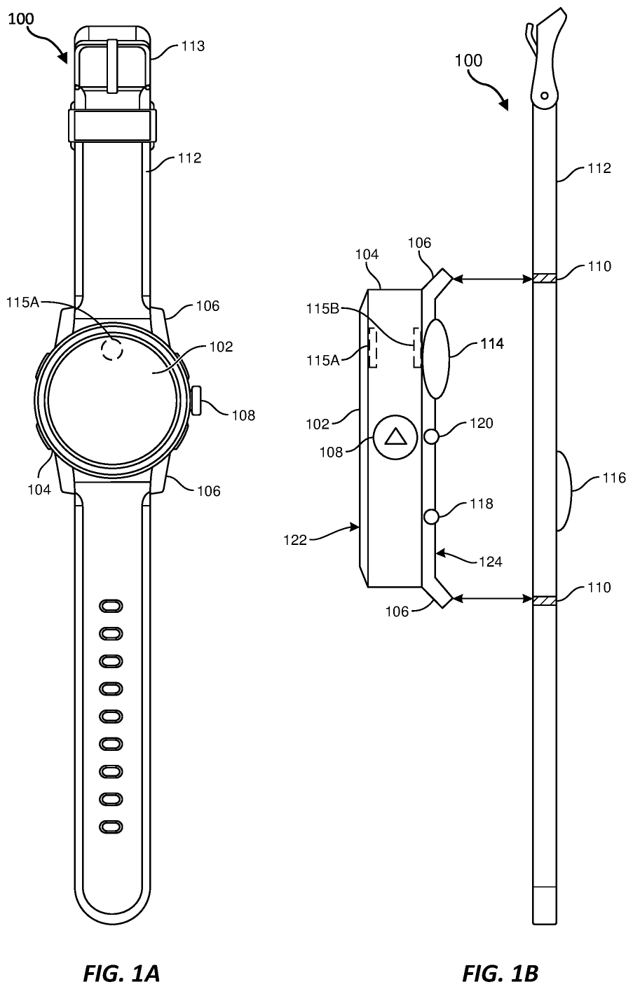

FIG. 1A illustrates an example wristband system 100 that includes a body 104 (e.g., a watch body, a display body, a wearable body, etc.) coupled to a band 112 (e.g., a watch band, a fitness tracker band, etc.). The wristband system 100 shown in FIGS. 1A and 1B is illustrated as a watch, and the body 104 and the band 112 are respectively referred to as the watch body 104 and the watch band 112. However, other wearable devices (e.g., arm bands, leg bands, bracelets, head bands, fitness trackers, etc.) are also possible implementations of the present disclosure.

The watch body 104 and watch band 112 may have any size and/or shape that is configured to allow a user to wear the wristband system 100 on a body part (e.g., a wrist). The watch body 104 may include a display screen 102 (e.g., a touch screen) and one or more buttons 108. The watch band 112 may include a retaining mechanism 113 (e.g., a buckle) for securing the watch band 112 to the user's wrist. The watch body 104 may also include a coupling mechanism 106 and the watch band 112 may include a corresponding coupling mechanism 110 for detachably coupling the watch body 104 to the watch band 112. A sensor 118 (e.g., a proximity sensor) may be configured for detecting whether the watch body 104 is coupled to or decoupled from the watch band 112.

The wristband system 100 may perform various functions associated with the user. The functions may be executed independently in the watch body 104, independently in the watch band 112, and/or jointly with the watch body 104 and the watch band 112 in communication with each other. Example functions that the wristband system 100 may execute may include, without limitation, heart rate monitoring, image capture, display of visual content to the user (e.g., via the display screen 102), sensing user input (e.g., sensing a touch on and/or press of the button 108, sensing a touch on the display screen 102, sensing biometric data with a biometric sensor 114, sensing neuromuscular signals with a neuromuscular sensor 116, etc.), messaging (e.g., text, speech, video messaging, etc.), image capture (e.g., with a front-facing image sensor 115A and/or a rear-facing image sensor 115B), wireless communications (e.g., cellular, near field, WiFi, personal area network communications, etc.), location determination, financial transactions, providing haptic feedback, etc. In some examples, the sensor 114 may include a HRM sensor. In some examples, the biometric sensor 114 may be integrated with the rear-facing image sensor 115B into a single package (e.g., a single multi-chip module package). In some examples, the biometric sensor 114 and rear-facing image sensor 115B may be separate components disposed in proximity to one another on a printed circuit board of the watch body 104. In some examples, functions may be executed on the wristband system 100 in conjunction with an AR system.

Although the wristband system 100 is described and shown as including the watch body 104 and watch band 112, the present disclosure is not limited to implementation via a wristwatch. Rather, in additional embodiments the wristband system 100 may be implemented as a fitness tracker, a bracelet, an arm band, a leg band, a necklace, a pendant, etc.

FIG. 1B illustrates the wristband system 100 with the watch body 104 decoupled from the watch band 112. The watch band 112 may be donned (e.g., worn) on a body part (e.g., a wrist) of a user and may operate independently from the watch body 104. For example, the watch band 112 may be configured to be worn by a user and an inner surface of the watch band 112 may be in contact with the user's skin. When the watch body 104 is coupled to the watch band 112 and worn by a user, the biometric sensor 114 may be in contact with the user's skin. The biometric sensor 114 may be or include a biosensor that senses a user's heart rate.

As noted above, the wristband system 100 may include a coupling mechanism 106, 110 for detachably coupling the watch body 104 to the watch band 112. A user may detach the watch body 104 from the watch band 112, such as to capture images using the rear-facing image sensor 115B, to charge the watch body 104, to switch watch bands 112, etc. Detaching the watch body 104 from the watch band 112 may reduce a physical profile and/or a weight of a portion of the wristband system 100 remaining on the user's wrist. Any sufficient method or coupling mechanism may be used for detachably coupling the watch body 104 to the watch band 112. By way of non-limiting examples, the coupling mechanism 106 may be or include magnets, a twist-to-lock and/or twist-to-unlock mechanism, a snap, a hook-and-loop fastener, an electronic pin actuator, a spring-loaded mechanism, etc.

As illustrated in FIG. 1B and as noted above, in some examples, the watch body 104 may include the front-facing image sensor 115A and the rear-facing image sensor 115B. The front-facing image sensor 115A may be located in a front face of the watch body 104 and the rear-facing image sensor 115B may be located in a rear face of the watch body 104. By way of example, a user may use the front-facing image sensor 115A to capture an image (e.g., a still image or a video) of the user, for a so-called “selfie view,” when the watch body 104 is attached to or detached from the watch band 112. When the watch body 104 is detached from the watch band 112, the user may use the rear-facing image sensor 115B to capture an image (e.g., a still image or a video) of a scene or object away from the user, for a so-called “world view.” The watch body 104 may include at least one LED 120. The at least one LED 120 may configured to provide a light source for the HRM sensor 114 when the watch body 104 is attached to the watch band 112. The at least one LED 120 may also be configured to provide a light source for the rear-facing image sensor 115B when the watch body 104 is not attached to the watch band 112.

The watch body 104 may include a front surface 122 and a rear surface 124 opposite the front surface 122. The display 102 and the front-facing image sensor 115A may be positioned on the front surface 122. The HRM sensor 114, rear-facing image sensor 115B, and at least one LED 120 may be positioned on the rear surface 124. The rear surface 124 may be configured to contact the user's wrist when the wristband system 100 is worn by the user with the watch body 104 attached to the watch band 112.

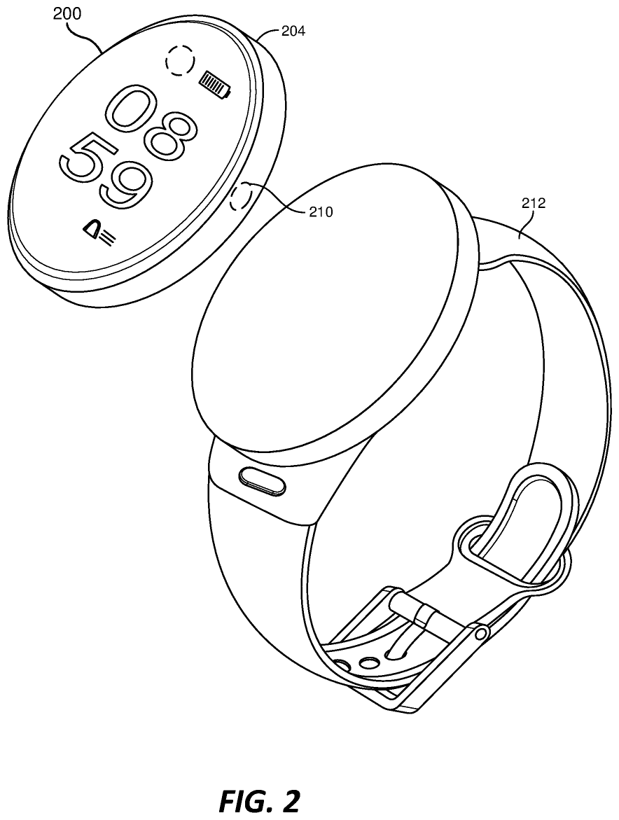

FIG. 2 illustrates a perspective view of an example wristband system 200 that includes a watch body 204 decoupled from a watch band 212. In some aspects, the wristband system 200 may be structured and/or function similarly to the wristband system 100 of FIGS. 1A and 1B. For example, the wristband system 200 may include a retaining mechanism (e.g., a buckle, a hook and loop fastener, etc.) for securing the watch band 212 to the user's wrist. The wristband system 200 may also include a coupling mechanism for detachably coupling the watch body 204 to the watch band 212. The wristband system 200 may include a coupling sensor 210 (e.g., a proximity sensor) configured to sense when the watch body 204 is coupled to and/or decoupled from the watch band 212. The coupling sensor 210 may include, without limitation, an inductive proximity sensor, a limit switch, an optical proximity sensor, a capacitive proximity sensor, a magnetic proximity sensor, an ultrasonic proximity sensor, or a combination thereof. A status of the coupling sensor 210 may be used to detect when the watch body 204 is coupled to the watch band 212 or decoupled from the watch band 212. The watch body 204 may include at least one LED, such as on a rear face of the watch body 204. The at least one LED may be configured to provide a light source for the HRM sensor when the watch body 204 is coupled to the watch band 212. The at least one LED may also be configured to provide a light source for a rear-facing image sensor when the watch body 204 is decoupled from the watch band 212.

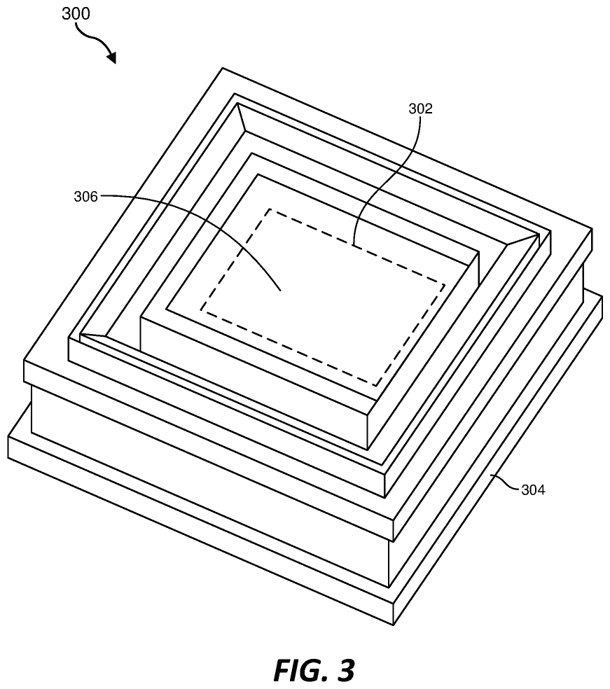

FIG. 3 is a perspective view of a single package 300 including an integrated flash LED and HRM LED 302 (also referred to herein as an “integrated LED 302”). In some examples, the integrated LED 302 may be integrated into the single package 300 for size, cost, and/or power consumption reduction. The package 300 may be used in a detachable watch body of a wristband system, such as any of the watch bodies 104, 204 discussed above. For example, when the watch body is detached from a corresponding watch band, the integrated LED 302 of the package 300 may provide a light source that may be used as a camera flash. When the watch body is attached to a watch band and against a user's skin, the integrated LED 302 of the package 300 may provide a light source for a HRM sensor.

In some examples, the package 300 may include a single integrated LED 302 that is configured to provide a light source for a HRM sensor and a light source (e.g., a flash LED) for rear-facing image sensor. The package 300 may include a multichip module on an interconnection substrate 304. The various components integrated into the package 300 may be mounted on the interconnection substrate 304 as bare dice and may be connected to a printed circuit board (PCB) within the watch body via wire bonding, tape bonding, or flip-chip bonding. The package 300 may be encapsulated by a plastic molding and mounted on a PCB within the watch body. The package 300 may also include an analog front end for processing analog signals.

In some examples, the integrated LED 302, when functioning as a camera flash, may include performance characteristics that may improve or facilitate image capture by an image sensor. The performance characteristics may include, without limitation, a color temperature range, a diagonal field of view range, a brightness level, a color rendering index, a uniformity level, and a rectangular illumination profile. The following discussion provides examples of suitable values for these performance characteristics, although different values may be used for a variety of configurations and applications.

For example, the integrated LED 302 may be capable of emitting light within a color temperature range of about D50 to about D60. D50 may be correlated to a color temperature of 5000 Kelvin. D60 may be correlated to a color temperature of 6000 Kelvin. The light emitted from the integrated LED 302 may include a diagonal field of view range of about 90 degrees. The integrated LED 302, when functioning as a camera flash, may emit light with a brightness level of at least 150 lux. The brightness level of at least 150 lux may be measured at a distance of 1 meter from the integrated LED 302 when the integrated LED 302 draws a current of 1 ampere. The integrated LED 302 may include a color rendering index (CRI) of about 80 CRI to about 90 CRI. The color rendering index may be a measurement of the color of light emitted from the integrated LED 302 when compared with sunlight. Light emitted from the integrated LED 302 may include a uniformity level of at least 30%. The uniformity level may be a ratio between a minimum brightness level emitted at a corner to a maximum brightness level emitted at a center of the integrated LED 302. The uniformity level of the integrated LED 302 may include a rectangular illumination profile (e.g., an aspect ratio) of about 4:3 to match the aspect ratio of the image sensor of a corresponding camera. The rectangular illumination profile may be a ratio of the width to height of the profile of light emitted by the integrated LED 302. In some examples, the rectangular illumination profile may be produced by a lens, such as a Fresnel lens. The performance characteristics of the integrated LED 302 may enable image capture that is of higher quality, compared to image capture by an image sensor that does not use the integrated LED 302 for illumination, particularly in low ambient light conditions.

The integrated LED 302 may include multiple components mounted on an interconnection substrate. The components may consume a volume on the interconnection substrate. The volume may include a range of dimensions. For example, the length of the volume may range from about 1.3 mm to about 4.0 mm. The width of the volume may range from about 1.0 mm to about 4.0 mm. The height of the volume may range from about 0.33 mm to about 1.4 mm. In some examples, the volume of the components may be correlated to the luminous output of the integrated LED 302. For example, the components having dimensions of about 2.0 mm in length, 1.6 mm in width, and 0.7 mm in height may be capable of generating light with a luminous output of about 300 lumens with a correlated color temperature of about 5000 Kelvin to 6000 Kelvin. The components having dimensions of about 1.6 mm in length, 1.0 mm in width, and 0.81 mm in height may have a luminous output of about 220 lumens with a correlated color temperature of about 5000 Kelvin to 6000 Kelvin. The components having dimensions of about 1.3 mm in length, 0.33 mm in width, and 0.7 mm in height may have a luminous output of about 280 lumens with a correlated color temperature of about 5000 Kelvin to 6000 Kelvin.

In some examples, the integrated LED 302 may include a lens 306 (e.g., a Fresnel lens, a total internal reflection (TIR) lens, etc.) mounted over the integrated LED 302 such that light emitted from the integrated LED 302 travels through the lens 306. The light traveling through the lens 306 may be focused, refracted, reflected, or a combination thereof. The lens 306 may capture and direct the emitted light photons to a desired location (e.g., towards an object to be illuminated). In some examples, the lens 306 may be configured and mounted over the integrated LED 302 such that an air gap exists between the integrated LED 302 and the lens 306. For example, the air gap may have a height in the range of about 0.01 mm to about 1.0 mm. The lens 306 may also include a lens opening size in the range of about 2.0 mm to about 3.0 mm. In some examples, a larger lens opening size may increase the optical efficiency of the combined lens 306 and integrated LED 302.

In some examples, the type of the lens 306 may influence a brightness of light emitted by the integrated LED 302. For example, a TIR lens may include an optical efficiency of about 60%. The TIR lens 306 may influence the brightness based on a distance from the TIR lens 306. When light of 260 lumens is illuminated through the TIR lens 306, about 380 lux may be detected at a distance of 0.5 mm. At a distance of 4.0 meters, about 6 lux may be detected. As another example, a Fresnel lens 306 may include an optical efficiency of about 30%. The Fresnel lens 306 may influence the brightness based on a distance from the Fresnel lens 306. When light of 260 lumens is illuminated through the Fresnel lens 306, about 216 lux may be detected at a distance of 0.5 mm. At a distance of 4.0 meters, about 3.4 lux may be detected.

As noted above, in some examples, the integrated LED 302 and supporting components may be mounted on a PCB. The dimensions for mounting the integrated LED 302 and supporting components on the PCB may be in a range of about 2.2 mm to about 3.2 mm in width and in a range of about 2.6 mm to about 3.2 mm in length.

In some examples, the integrated LED 302 may consume a variable amount of electrical current during periods of operation. For example, when used as a camera flash, the integrated LED 302 may operate during a pre-flash period. The pre-flash period may be a time period of about 350 milliseconds. During the pre-flash period, the integrated LED 302 may consume a relatively lower amount of electrical current, such as about 360 milliamps. During the pre-flash period, charge storage devices (e.g., capacitors) may be charged. During the flash period, when light is emitted from the integrated LED 302 to illuminate a scene, the integrated LED 302 may consume a relatively higher amount of electrical current, such as about 1 ampere. Current may be supplied to the integrated LED 302 by an LED driver. The LED driver may exhibit an efficiency in the range of about 80% to about 82%. The LED driver efficiency may be a ratio of the amount of current supplied to the integrated LED 302 by the LED driver to the amount of current supplied to the LED driver by a battery or other power source. In some examples, the execution of about 502 cycles of pre-flash periods and flash periods may use about 10% of the battery capacity. In some examples, executing about 5024 cycles of pre-flash periods and flash periods may use about 100% of the battery capacity.

In some examples, the cycling of the integrated LED 302 by the LED driver may increase the amount of heat generated in the integrated LED 302 and/or the LED driver. In order to ensure that the junction temperature (Tⱼ) of semiconductor components included in the package 300 remain below a threshold, heat dissipation components may be employed to dissipate heat away from the semiconductor components. The junction temperature may be the maximum temperature that the semiconductor components can tolerate to ensure reliable operation. For example, heat sink component(s) may be configured to draw heat away from the semiconductor components and keep the junction temperature of the semiconductor components within an acceptable temperature range.

Any type of heat sink component(s) may be configured to draw heat away from the semiconductor components to dissipate the heat and allow regulation of the temperature of the semiconductor component(s). For example, thermally conductive material(s) (e.g., metal, aluminum, copper, gold, tin, aluminum nitride substrate, etc.) may be configured to draw heat away from the semiconductor component(s). The thermally conductive material may be configured as metal clips that are disposed between the integrated LED 302 and/or the LED driver package and a PCB on which the integrated LED 302 and/or the LED driver package are mounted. In some examples, the metal clips may transfer the heat to the printed circuit board for dissipation through natural convection methods. The heat dissipation devices and methods described above may keep the junction temperature of the semiconductor components under a junction temperature threshold. By way of example and not limitation, during cycling of the integrated LED 302 and the LED driver, the junction temperature may be kept below a threshold of 100 degrees Celsius.

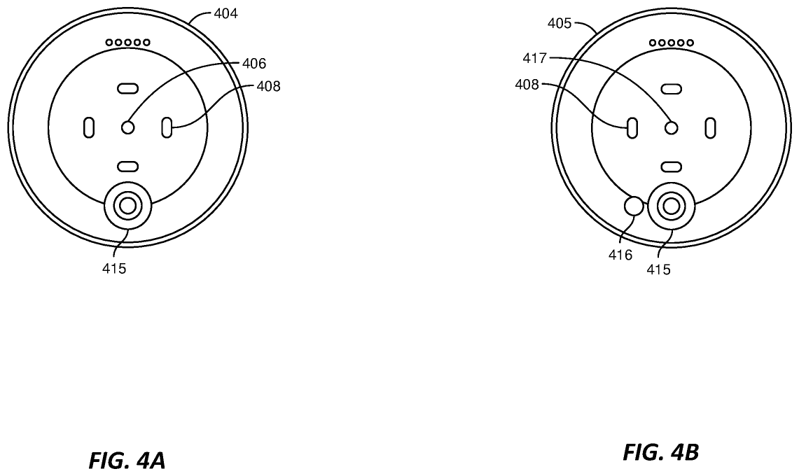

FIG. 4A illustrates a plan view of an example watch body 404 with an integrated flash LED and HRM LED 406 (also referred to herein as “integrated LED 406”) and a rear-facing image sensor 415. In some examples, the integrated LED 406 may be mounted on a rear face of watch body 404 such that the integrated LED 406 is adjacent to (e.g., contacts, nearly contacts, etc.) a surface of the user's skin when the watch body 404 is worn by the user (e.g., with a corresponding watch band). The integrated LED 406 may be positioned (e.g., in the center of the watch body 404) such that light from the integrated LED 406 is exposed to the user's skin for heart rate monitoring. Light sensors 408 (e.g., four light sensors 408) may also be positioned on the rear face of the watch body 404 to receive light emitted by the integrated LED 406 and reflected from the user's body (e.g., arm) for sensing the user's heart rate.

When the watch body 404 is not worn by the user, such as when the watch body 404 is decoupled from a corresponding watch band, light from the integrated LED 406 may be configured to illuminate an area that may be captured by the rear-facing image sensor 415. In this manner, the integrated LED 406 may include a single LED package that performs functions for both heart rate monitoring and illuminating a scene for image capture.

In some examples, the rear-facing image sensor 415 may not share a same window as the integrated LED 406 in order to reduce light leakage between the integrated LED 406 and the rear-facing image sensor 415. Thus, a window of the rear-facing image sensor 415 may be separate and isolated from a window of the integrated LED 406.

FIG. 4B illustrates a plan view of an example watch body 405 with a separate flash LED 416 and HRM LED 417 disposed on a rear face of the watch body 405. In some respects, the watch body 405 of FIG. 4B may be similar to the watch body 404 discussed above with reference to FIG. 4A. For example, the watch body 405 may include a rear-facing image sensor 415 that is configured to capture images when the watch body 405 is not attached to a corresponding watch band. As shown in FIG. 4B, the watch body 405 may also include an HRM LED 417 positioned (e.g., in the center of the watch body 405) such that light from the HRM LED 417 is exposed to the user's skin when the watch body 405 is worn by the user. Light sensors 408 (e.g., photodiodes) for heart rate monitoring may be disposed in the watch body 405 and may be configured to sense light emitted by the HRM LED 417 and reflected from the user (e.g., reflected and/or scattered by the user's blood to perform pulse oximetry).

In some examples, the flash LED 416 may be positioned away from the center of the watch body 405 (e.g., towards an edge of the watch body 405) such that light from the flash LED 416 may illuminate an area that may be captured by the rear-facing image sensor 415. In some examples, the light from the flash LED 416 may have a field of view that is narrower compared to the embodiment of FIG. 4A based on the position of the flash LED 416 relative to the position of the rear-facing image sensor 415.

Referring to FIGS. 4A and 4B, any of the integrated LED 406, flash LED 416, and/or HRM LED 417 (referred to collectively as the “LEDs 406, 416, 417”) may be configured to emit light at a variety of different wavelengths and/or visible colors. For example, one or more of the LEDs 406, 416, 417 may include light sources capable of emitting infrared light, white light, red light, green light, blue light, amber light, cyan light, magenta light, yellow light, or any combination thereof, including other colors that may be produced by combining two or more colors. By way of example and not limitation, one or more of the LEDs 406, 416, 417 may include a red-green-blue (RGB) LED package, a cyan-magenta-yellow (CYM) LED package, an infrared (IR) LED package, a combination RGB and IR LED package, a combination CYM and IR LED package, etc. In additional embodiments, any of the LEDs 406, 416, 417 may be or include a vertical cavity surface emitting laser (VCSEL) LED.

The watch body 404, 405 may include a proximity sensor configured to sense when the watch body 404, 405 is attached to a corresponding watch band, against the user's skin, and/or against another solid surface (e.g., a table). For example, in embodiments that include one or more of the LEDs 406, 416, 417 capable of emitting IR light, the IR light may be used for proximity sensing. IR light emitted from one or more of the LEDs 406, 416, 417 may reflect off nearby surfaces, such as the user's skin or another surface. The reflected IR light may be sensed by an IR sensor in the watch body 404, 405 (e.g., through one or more of the light sensors 408) to determine the distance between the watch body 404, 405 and the nearby surface.

Proximity sensing between the watch body 404, 405 and a nearby surface may be useful in a variety of ways. For example, the proximity sensing may be useful for security and privacy. When the watch body 404, 405 senses that it is away from the user's wrist, certain functions (e.g., digital payments, accessing sensitive content stored on displayed by the watch body 404, 405, viewing messages, sending messages, making calls, etc.) may be deactivated, locked, or otherwise made unavailable without entry of a passcode, biometric data, or other form of authentication. Thus, another person who is not an owner of the watch body 404, 405 may be prohibited from using at least certain functions of the watch body 404, 405 when the watch body 404, 405 is detached from a corresponding watch band and/or when not worn by the user.

In additional embodiments, the proximity sensing may be useful for power management. For example, when the watch body 404, 405 senses detachment, certain functions that are primarily useful when the watch body 404, 405 is worn may be deactivated or placed in a low-power mode. Such functions may include heart rate monitoring, fitness tracking (e.g., step counting, etc.), pulse oximetry, sleep tracking, etc.

Referring again to FIGS. 4A and 4B, in some embodiments, any of the LEDs 406, 416, 417 may be configured to serve as a visual privacy indicator. For example, when the watch body 404 or 405 is detached from a corresponding watch band, the rear-facing image sensor 415 may be used to capture an image and/or record a video. When an image is being taken or a video is being recorded, one or more of the LEDs 406, 416, 417 may provide a visual indication, such as a flashing light of any suitable color (e.g., green, red, yellow, blue, white, orange, etc.). This privacy indication may alert a person who is in view of the rear-facing image sensor 415 that an image and/or video is being recorded. Indications other than for privacy may also be provided by any of the LEDs 406, 416, 417, such as a low-battery indicator, a fully-charged battery indicator, an incoming message indicator, etc. In additional embodiments, any of the LEDs 406, 416, 417 may be used as a flashlight.

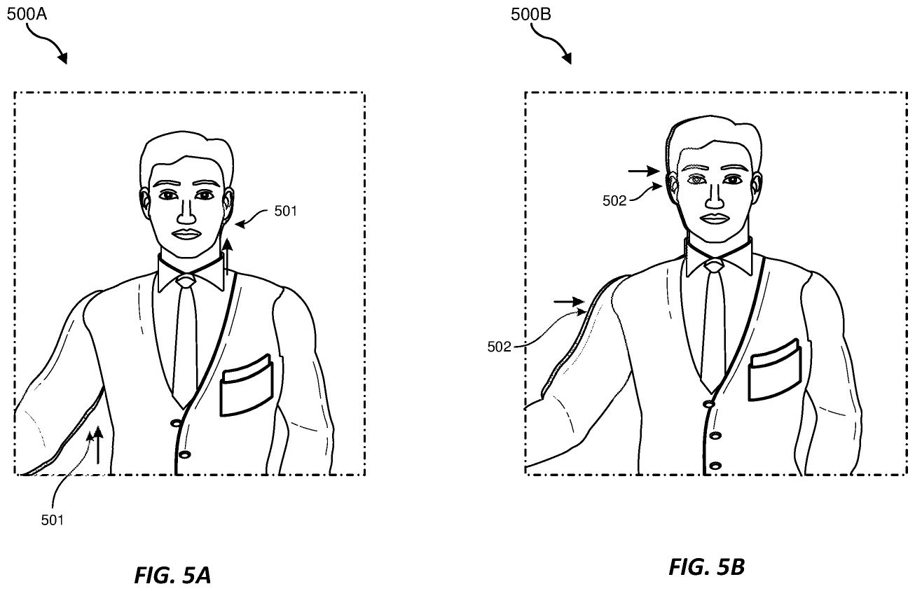

FIGS. 5A and 5B illustrate shadows introduced during the capture of an image 500A, 500B as a result of two respective flash LED configurations. In some examples, a shadow may be introduced into an image captured by an image sensor based on the configuration of a window (e.g., a lens) that covers the flash LED.

For example, FIG. 5A shows that shadows 501 may be introduced into an image 500A of a person captured by an image sensor when the flash LED and the HRM LED are integrated into a single LED package and/or share the same window (e.g., share the same lens), such as in the configuration shown in FIG. 4A. For example, the shadow 501 may be introduced into the image 500A of the person under the person's left ear and/or right arm. As another example, FIG. 5B shows that shadows 502 may be introduced into an image 500B of a person captured by an image sensor when the flash LED and the HRM LED each have a separate window (e.g., a separate lens for the flash LED and the HRM LED), such as the configuration shown in FIG. 4B. For example, a shadow 502 may be introduced into the image 500B of the person on the right side of the person's arm and/or the right side of the person's head. Thus, the configuration and placement of the flash LED relative to an image sensor may influence shadow placement in resulting images 500A, 500B.

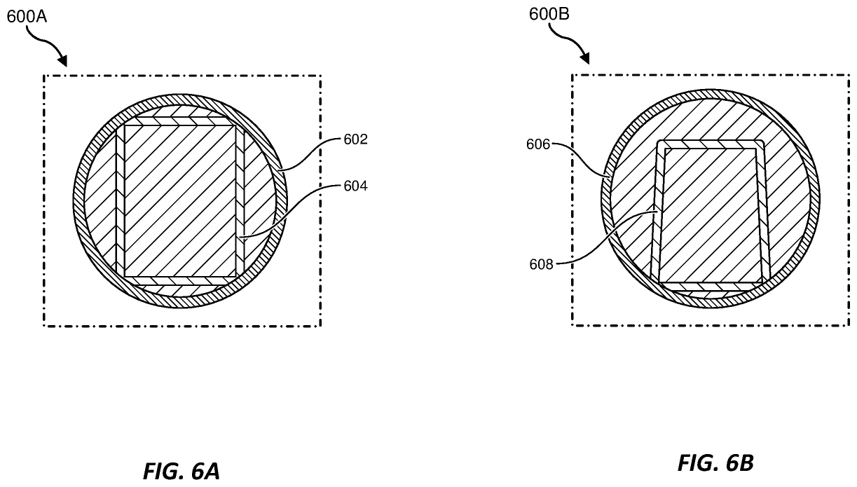

FIGS. 6A and 6B are diagrams 600A, 600B illustrating fields of view of a flash LED in various configurations. In some examples, the flash LED may be configured to have a field of view covers an image capture area with tilt tolerances. For example, FIG. 6A shows a field of view of the flash LED without tilt tolerances, whereas FIG. 6B shows the field of view of the flash LED with manufacturing tilt tolerances.

Referring to FIG. 6A, an area 602 illuminated by the flash LED completely covers an image capture area 604. In this case, there is little or no tilt tolerance, meaning that a flash LED and image capture device should be installed with alignment in their fields of view to ensure full illumination of the image capture area 604.

Referring to FIG. 6B, an area 606 illuminated by the flash LED may completely cover an image capture area 608. In this case, there is a tilt tolerance because the area 606 covers an area that is larger than the image capture area 608 with a margin. By way of example and not limitation, the manufacturing tilt tolerances may include about +/−3 degree tilt for the camera and about +/−3 degree tilt for the flash LED. Thus, the placement angle of these components need not be as precise as in the embodiment of FIG. 6A.

In an embodiment in which the flash LED and the HRM LED share the same window (e.g., share the same lens), such as the configuration shown in FIG. 4A, the field of view without tilt tolerances (FIG. 6A) may be about 80.6 degrees. With manufacturing tilt tolerances (FIG. 6B), the field of view may be about 90.5 degrees. In the embodiment in which the flash LED and the HRM LED each have a separate window (e.g., a separate lens for the flash LED and HRM LED), such as the configuration shown in FIG. 4B, the field of view without tilt tolerances (FIG. 6A) may be about 80.3 degrees. With manufacturing tilt tolerances (FIG. 6B), the field of view may be about 90.1 degrees.

The flash LED may have a non-uniform illuminance distribution within the field of view. The illuminance may be highest within a center portion of the field of view and decrease with distance from the center. For example, in an embodiment in which the flash LED is a single-color LED covered by a TIR lens, the illuminance at the corner of the field of view may be about 30% of the illuminance at the center of the field of view. In an embodiment in which the flash LED is a dual color LED (e.g., a warm white LED and a cool white LED) covered by a Fresnel lens, the illuminance distribution across the field of view may be more uniform than the illuminance distribution across the field of view of a single color flash LED. In some examples, multiple LEDs having different color temperatures may be used to create a specific color temperature (e.g., by driving the LEDs at different intensity levels), based on ambient lighting conditions.

In some examples, the flash LED may be characterized based on illumination parameters. The illumination parameters may include, without limitation, luminous energy, luminous flux, luminous intensity, luminance, illuminance, or a combination thereof. In some examples, the flash LED may be characterized based on operating parameters. The operating parameters may include, without limitation, power dissipation, pulsed forward current, junction temperature, electrostatic discharge threshold, operating temperature range, storage temperature range, viewing angle, color temperature, forward voltage, reverse current, or a combination thereof.

In order to capture high-quality images, the flash LED may meet a minimum illumination target value at a set distance from the flash LED. Additionally or alternatively, the flash LED may illuminate a sequence of flashes. For example, an initial flash may include a low-level torch mode that enables tuning of an auto-focus function. A subsequent flash may include a pre-flash of similar energy as the main flash to determine the best exposure under flash conditions. A final flash may include a short time period flash within frame blanking.

In some examples, an HRM may include multiple components configured as a system-in-package (SIP). The SIP may include, without limitation, an analog front end, at least one photodiode, and at least one HRM LED. The at least one HRM LED may include one or more LEDs of a various colors and types as discussed above, such as, without limitation, a green LED, a red LED, a blue LED, an infrared LED, a VCSEL LED, or a combination thereof. The SIP may be configured to be installed on a PCB as a single component. The advantages of integrating the HRM components into a SIP include a smaller volume compared to a discrete solution, and an optimized, tested, and proven design.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

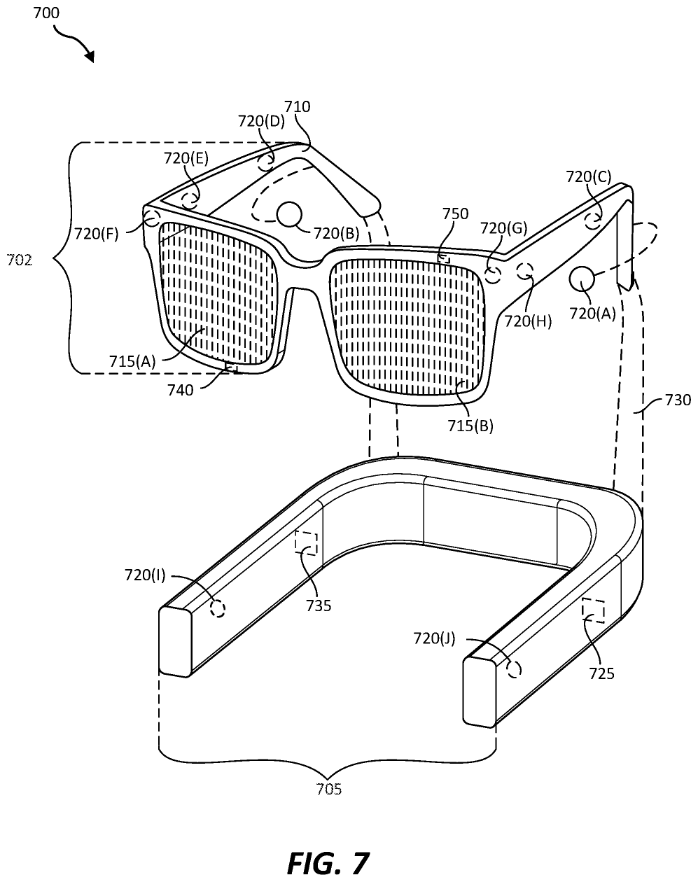

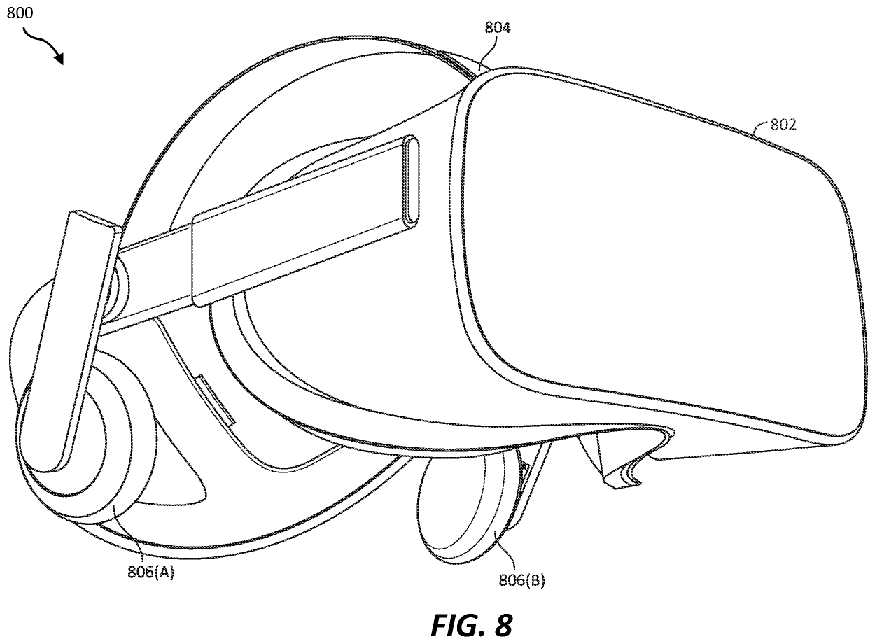

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., an augmented-reality system 700 in FIG. 7) or that visually immerses a user in an artificial reality (such as, e.g., a virtual-reality system 800 in FIG. 8). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 7, the augmented-reality system 700 may include an eyewear device 702 with a frame 710 configured to hold a left display device 715(A) and a right display device 715(B) in front of a user's eyes. The display devices 715(A) and 715(B) may act together or independently to present an image or series of images to a user. While the augmented-reality system 700 includes two displays, embodiments of this disclosure may be implemented in augmented-reality systems with a single NED or more than two NEDs.

In some embodiments, the augmented-reality system 700 may include one or more sensors, such as a sensor 740. The sensor 740 may generate measurement signals in response to motion of the augmented-reality system 700 and may be located on substantially any portion of the frame 710. The sensor 740 may represent one or more of a variety of different sensing mechanisms, such as a position sensor, an inertial measurement unit (IMU), a depth camera assembly, a structured light emitter and/or detector, or any combination thereof. In some embodiments, the augmented-reality system 700 may or may not include the sensor 740 or may include more than one sensor. In embodiments in which the sensor 740 includes an IMU, the IMU may generate calibration data based on measurement signals from the sensor 740. Examples of the sensor 740 may include, without limitation, accelerometers, gyroscopes, magnetometers, other suitable types of sensors that detect motion, sensors used for error correction of the IMU, or some combination thereof.

In some examples, the augmented-reality system 700 may also include a microphone array with a plurality of acoustic transducers 720(A)-720(J), referred to collectively as acoustic transducers 720. The acoustic transducers 720 may represent transducers that detect air pressure variations induced by sound waves. Each acoustic transducer 720 may be configured to detect sound and convert the detected sound into an electronic format (e.g., an analog or digital format). The microphone array in FIG. 7 may include, for example, ten acoustic transducers: 720(A) and 720(B), which may be designed to be placed inside a corresponding ear of the user, acoustic transducers 720(C), 720(D), 720(E), 720(F), 720(G), and 720(H), which may be positioned at various locations on the frame 710, and/or acoustic transducers 720(l) and 720(J), which may be positioned on a corresponding neckband 705.

In some embodiments, one or more of the acoustic transducers 720(A)-(J) may be used as output transducers (e.g., speakers). For example, the acoustic transducers 720(A) and/or 720(B) may be earbuds or any other suitable type of headphone or speaker.

The configuration of the acoustic transducers 720 of the microphone array may vary. While the augmented-reality system 700 is shown in FIG. 7 as having ten acoustic transducers 720, the number of acoustic transducers 720 may be greater or less than ten. In some embodiments, using higher numbers of acoustic transducers 720 may increase the amount of audio information collected and/or the sensitivity and accuracy of the audio information. In contrast, using a lower number of acoustic transducers 720 may decrease the computing power required by an associated controller 750 to process the collected audio information. In addition, the position of each acoustic transducer 720 of the microphone array may vary. For example, the position of an acoustic transducer 720 may include a defined position on the user, a defined coordinate on the frame 710, an orientation associated with each acoustic transducer 720, or some combination thereof.

The acoustic transducers 720(A) and 720(B) may be positioned on different parts of the user's ear, such as behind the pinna, behind the tragus, and/or within the auricle or fossa. Or, there may be additional acoustic transducers 720 on or surrounding the ear in addition to the acoustic transducers 720 inside the ear canal. Having an acoustic transducer 720 positioned next to an ear canal of a user may enable the microphone array to collect information on how sounds arrive at the ear canal. By positioning at least two of the acoustic transducers 720 on either side of a user's head (e.g., as binaural microphones), the augmented-reality device 700 may simulate binaural hearing and capture a 3D stereo sound field around about a user's head. In some embodiments, the acoustic transducers 720(A) and 720(B) may be connected to the augmented-reality system 700 via a wired connection 730, and in other embodiments the acoustic transducers 720(A) and 720(B) may be connected to the augmented-reality system 700 via a wireless connection (e.g., a Bluetooth connection). In still other embodiments, the acoustic transducers 720(A) and 720(B) may not be used at all in conjunction with the augmented-reality system 700.

The acoustic transducers 720 on the frame 710 may be positioned in a variety of different ways, including along the length of the temples, across the bridge, above or below the display devices 715(A) and 715(B), or some combination thereof. The acoustic transducers 720 may also be oriented such that the microphone array is able to detect sounds in a wide range of directions surrounding the user wearing the augmented-reality system 700. In some embodiments, an optimization process may be performed during manufacturing of the augmented-reality system 700 to determine relative positioning of each acoustic transducer 720 in the microphone array.

In some examples, the augmented-reality system 700 may include or be connected to an external device (e.g., a paired device), such as the neckband 705. The neckband 705 generally represents any type or form of paired device. Thus, the following discussion of the neckband 705 may also apply to various other paired devices, such as charging cases, smart watches, smart phones, wristbands, other wearable devices, hand-held controllers, tablet computers, laptop computers, other external compute devices, etc.

As shown, the neckband 705 may be coupled to the eyewear device 702 via one or more connectors. The connectors may be wired or wireless and may include electrical and/or non-electrical (e.g., structural) components. In some cases, the eyewear device 702 and neckband 705 may operate independently without any wired or wireless connection between them. While FIG. 7 illustrates the components of the eyewear device 702 and neckband 705 in example locations on the eyewear device 702 and neckband 705, the components may be located elsewhere and/or distributed differently on the eyewear device 702 and/or neckband 705. In some embodiments, the components of the eyewear device 702 and neckband 705 may be located on one or more additional peripheral devices paired with the eyewear device 702, the neckband 705, or some combination thereof.

Pairing external devices, such as the neckband 705, with augmented-reality eyewear devices may enable the eyewear devices to achieve the form factor of a pair of glasses while still providing sufficient battery and computation power for expanded capabilities. Some or all of the battery power, computational resources, and/or additional features of the augmented-reality system 700 may be provided by a paired device or shared between a paired device and an eyewear device, thus reducing the weight, heat profile, and form factor of the eyewear device overall while still retaining desired functionality. For example, the neckband 705 may allow components that would otherwise be included on an eyewear device to be included in the neckband 705 since users may tolerate a heavier weight load on their shoulders than they would tolerate on their heads. The neckband 705 may also have a larger surface area over which to diffuse and disperse heat to the ambient environment. Thus, the neckband 705 may allow for greater battery and computation capacity than might otherwise have been possible on a stand-alone eyewear device. Since weight carried in the neckband 705 may be less invasive to a user than weight carried in the eyewear device 702, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

The neckband 705 may be communicatively coupled with the eyewear device 702 and/or to other devices. These other devices may provide certain functions (e.g., tracking, localizing, depth mapping, processing, storage, etc.) to the augmented-reality system 700. In the embodiment of FIG. 7, the neckband 705 may include two acoustic transducers (e.g., 720(l) and 720(J)) that are part of the microphone array (or potentially form their own microphone subarray). The neckband 705 may also include a controller 725 and a power source 735.

Acoustic transducers 720(1) and 720(J) of the neckband 705 may be configured to detect sound and convert the detected sound into an electronic format (analog or digital). In the embodiment of FIG. 7, the acoustic transducers 720(1) and 720(J) may be positioned on the neckband 705, thereby increasing the distance between the neckband acoustic transducers 720(l) and 720(J) and other acoustic transducers 720 positioned on the eyewear device 702. In some cases, increasing the distance between the acoustic transducers 720 of the microphone array may improve the accuracy of beamforming performed via the microphone array. For example, if a sound is detected by the acoustic transducers 720(C) and 720(D) and the distance between the acoustic transducers 720(C) and 720(D) is greater than, e.g., the distance between the acoustic transducers 720(D) and 720(E), the determined source location of the detected sound may be more accurate than if the sound had been detected by the acoustic transducers 720(D) and 720(E).

The controller 725 of the neckband 705 may process information generated by the sensors on the neckband 705 and/or the augmented-reality system 700. For example, the controller 725 may process information from the microphone array that describes sounds detected by the microphone array. For each detected sound, the controller 725 may perform a direction-of-arrival (DOA) estimation to estimate a direction from which the detected sound arrived at the microphone array. As the microphone array detects sounds, the controller 725 may populate an audio data set with the information. In embodiments in which the augmented-reality system 700 includes an inertial measurement unit, the controller 725 may compute all inertial and spatial calculations from the IMU located on the eyewear device 702. A connector may convey information between the augmented-reality system 700 and the neckband 705 and between the augmented-reality system 700 and the controller 725. The information may be in the form of optical data, electrical data, wireless data, or any other transmittable data form. Moving the processing of information generated by the augmented-reality system 700 to the neckband 705 may reduce weight and heat in the eyewear device 702, making it more comfortable to the user.

The power source 735 in the neckband 705 may provide power to the eyewear device 702 and/or to the neckband 705. The power source 735 may include, without limitation, lithium-ion batteries, lithium-polymer batteries, primary lithium batteries, alkaline batteries, or any other form of power storage. In some cases, the power source 735 may be a wired power source. Including the power source 735 on the neckband 705 instead of on the eyewear device 702 may help better distribute the weight and heat generated by the power source 735.

As noted, some artificial-reality systems may, instead of blending an artificial reality with actual reality, substantially replace one or more of a user's sensory perceptions of the real world with a virtual experience. One example of this type of system is a head-worn display system, such as the virtual-reality system 800 in FIG. 8, that mostly or completely covers a user's field of view. The virtual-reality system 800 may include a front rigid body 802 and a band 804 shaped to fit around a user's head. The virtual-reality system 800 may also include output audio transducers 806(A) and 806(B). Furthermore, while not shown in FIG. 8, the front rigid body 802 may include one or more electronic elements, including one or more electronic displays, one or more inertial measurement units (IMUs), one or more tracking emitters or detectors, and/or any other suitable device or system for creating an artificial-reality experience.

Artificial-reality systems may include a variety of types of visual feedback mechanisms. For example, display devices in the augmented-reality system 700 and/or the virtual-reality system 800 may include one or more liquid crystal displays (LCDs), light emitting diode (LED) displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., conventional concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in the augmented-reality system 700 and/or the virtual-reality system 800 may include micro-LED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, the augmented-reality system 700 and/or the virtual-reality system 800 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (e.g., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

As noted, the artificial-reality systems 700 and 800 may be used with a variety of other types of devices to provide a more compelling artificial-reality experience. These devices may be haptic interfaces with transducers that provide haptic feedback and/or that collect haptic information about a user's interaction with an environment. The artificial-reality systems disclosed herein may include various types of haptic interfaces that detect or convey various types of haptic information, including tactile feedback (e.g., feedback that a user detects via nerves in the skin, which may also be referred to as cutaneous feedback) and/or kinesthetic feedback (e.g., feedback that a user detects via receptors located in muscles, joints, and/or tendons).

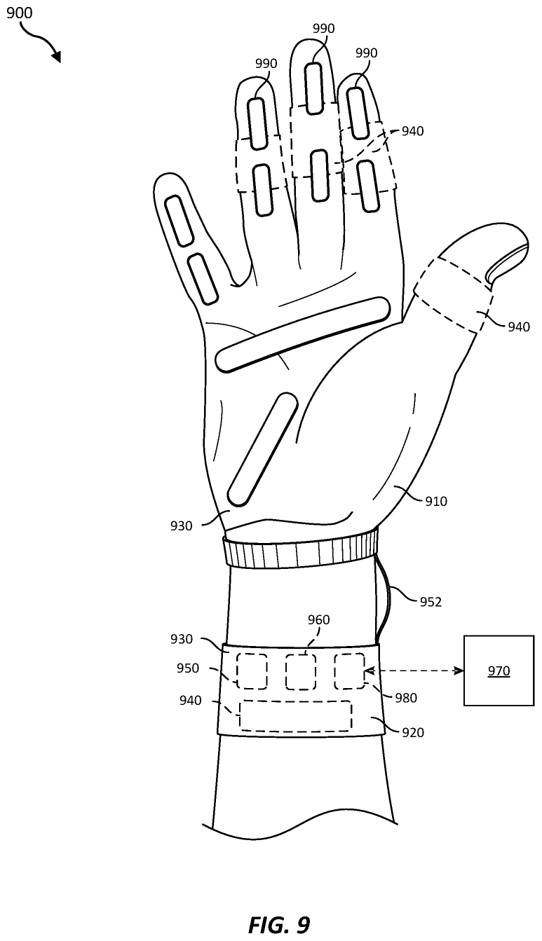

Haptic feedback may be provided by interfaces positioned within a user's environment (e.g., chairs, tables, floors, etc.) and/or interfaces on articles that may be worn or carried by a user (e.g., gloves, wristbands, etc.). As an example, FIG. 9 illustrates a vibrotactile system 900 in the form of a wearable glove (haptic device 910) and wristband (haptic device 920). The haptic device 910 and the haptic device 920 are shown as examples of wearable devices that include a flexible, wearable textile material 930 that is shaped and configured for positioning against a user's hand and wrist, respectively. This disclosure also includes vibrotactile systems that may be shaped and configured for positioning against other human body parts, such as a finger, an arm, a head, a torso, a foot, or a leg. By way of example and not limitation, vibrotactile systems according to various embodiments of the present disclosure may also be in the form of a glove, a headband, an armband, a sleeve, a head covering, a sock, a shirt, or pants, among other possibilities. In some examples, the term “textile” may include any flexible, wearable material, including woven fabric, non-woven fabric, leather, cloth, a flexible polymer material, composite materials, etc.

One or more vibrotactile devices 940 may be positioned at least partially within one or more corresponding pockets formed in the textile material 930 of the vibrotactile system 900. The vibrotactile devices 940 may be positioned in locations to provide a vibrating sensation (e.g., haptic feedback) to a user of the vibrotactile system 900. For example, the vibrotactile devices 940 may be positioned against the user's finger(s), thumb, or wrist, as shown in FIG. 9. The vibrotactile devices 940 may, in some examples, be sufficiently flexible to conform to or bend with the user's corresponding body part(s).

A power source 950 (e.g., a battery) for applying a voltage to the vibrotactile devices 940 for activation thereof may be electrically coupled to the vibrotactile devices 940, such as via conductive wiring 952. In some examples, each of the vibrotactile devices 940 may be independently electrically coupled to the power source 950 for individual activation. In some embodiments, a processor 960 may be operatively coupled to the power source 950 and configured (e.g., programmed) to control activation of the vibrotactile devices 940.

The vibrotactile system 900 may be implemented in a variety of ways. In some examples, the vibrotactile system 900 may be a standalone system with integral subsystems and components for operation independent of other devices and systems. As another example, the vibrotactile system 900 may be configured for interaction with another device or system 970. For example, the vibrotactile system 900 may include a communications interface 980 for receiving and/or sending signals to the other device or system 970. The other device or system 970 may be a mobile device, a gaming console, an artificial-reality (e.g., virtual-reality, augmented-reality, mixed-reality) device, a personal computer, a tablet computer, a network device (e.g., a modem, a router, etc.), a handheld controller, etc. The communications interface 980 may enable communications between the vibrotactile system 900 and the other device or system 970 via a wireless (e.g., Wi-Fi, Bluetooth, cellular, radio, etc.) link or a wired link. If present, the communications interface 980 may be in communication with the processor 960, such as to provide a signal to the processor 960 to activate or deactivate one or more of the vibrotactile devices 940.

The vibrotactile system 900 may optionally include other subsystems and components, such as touch-sensitive pads 990, pressure sensors, motion sensors, position sensors, lighting elements, and/or user interface elements (e.g., an on/off button, a vibration control element, etc.). During use, the vibrotactile devices 940 may be configured to be activated for a variety of different reasons, such as in response to the user's interaction with user interface elements, a signal from the motion or position sensors, a signal from the touch-sensitive pads 990, a signal from the pressure sensors, a signal from the other device or system 970, etc.

Although the power source 950, processor 960, and communications interface 980 are illustrated in FIG. 9 as being positioned in the haptic device 920, the present disclosure is not so limited. For example, one or more of the power source 950, processor 960, or communications interface 980 may be positioned within the haptic device 910 or within another wearable textile.

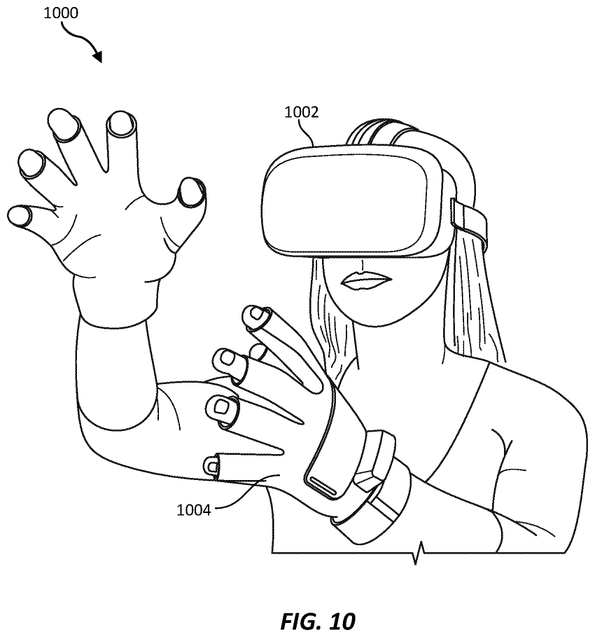

Haptic wearables, such as those shown in and described in connection with FIG. 9, may be implemented in a variety of types of artificial-reality systems and environments. FIG. 10 shows an example artificial-reality environment 1000 including one head-mounted virtual-reality display and two haptic devices (e.g., gloves), and in other embodiments any number and/or combination of these components and other components may be included in an artificial-reality system. For example, in some embodiments there may be multiple head-mounted displays each having an associated haptic device, with each head-mounted display and each haptic device communicating with the same console, portable computing device, or other computing system.

A head-mounted display 1002, as depicted in FIG. 10, generally represents any type or form of virtual-reality system, such as the virtual-reality system 800 in FIG. 8. Haptic device 1004 generally represents any type or form of wearable device, worn by a user of an artificial-reality system, that provides haptic feedback to the user to give the user the perception that he or she is physically engaging with a virtual object. In some embodiments, the haptic device 1004 may provide haptic feedback by applying vibration, motion, and/or force to the user. For example, the haptic device 1004 may limit or augment a user's movement. To give a specific example, the haptic device 1004 may limit a user's hand from moving forward so that the user has the perception that his or her hand has come in physical contact with a virtual wall. In this specific example, one or more actuators within the haptic device may achieve the physical-movement restriction by pumping fluid into an inflatable bladder of the haptic device. In some examples, a user may also use the haptic device 1004 to send action requests to a console. Examples of action requests include, without limitation, requests to start an application and/or end the application and/or requests to perform a particular action within the application.

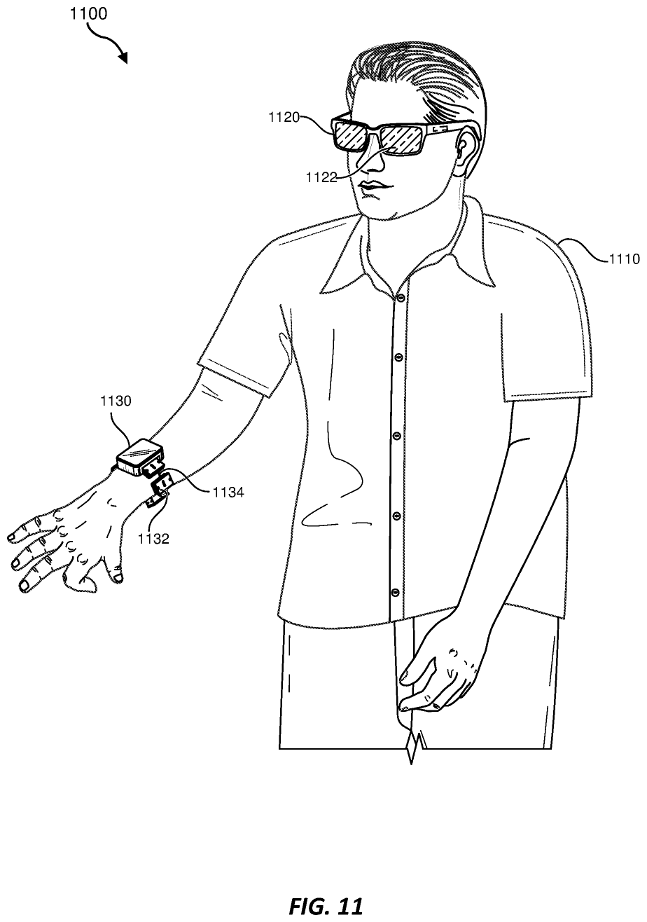

While haptic interfaces may be used with virtual-reality systems, as shown in FIG. 10, haptic interfaces may also be used with augmented-reality systems, as shown in FIG. 11. FIG. 11 is a perspective view of a user 1110 interacting with an augmented-reality system 1100. In this example, the user 1110 may wear a pair of augmented-reality glasses 1120 that may have one or more displays 1122 and that are paired with a haptic device 1130. In this example, the haptic device 1130 may be a wristband that includes a plurality of band elements 1132 and a tensioning mechanism 1134 that connects band elements 1132 to one another.

One or more of the band elements 1132 may include any type or form of actuator suitable for providing haptic feedback. For example, one or more of the band elements 1132 may be configured to provide one or more of various types of cutaneous feedback, including vibration, force, traction, texture, and/or temperature. To provide such feedback, the band elements 1132 may include one or more of various types of actuators. In one example, each of the band elements 1132 may include a vibrotactor (e.g., a vibrotactile actuator) configured to vibrate in unison or independently to provide one or more of various types of haptic sensations to a user. Alternatively, only a single band element or a subset of band elements may include vibrotactors.

The haptic devices 910, 920, 1004, and 1130 may include any suitable number and/or type of haptic transducer, sensor, and/or feedback mechanism. For example, the haptic devices 910, 920, 1004, and 1130 may include one or more mechanical transducers, piezoelectric transducers, and/or fluidic transducers. The haptic devices 910, 920, 1004, and 1130 may also include various combinations of different types and forms of transducers that work together or independently to enhance a user's artificial-reality experience. In one example, each of the band elements 1132 of the haptic device 1130 may include a vibrotactor (e.g., a vibrotactile actuator) configured to vibrate in unison or independently to provide one or more of various types of haptic sensations to a user.

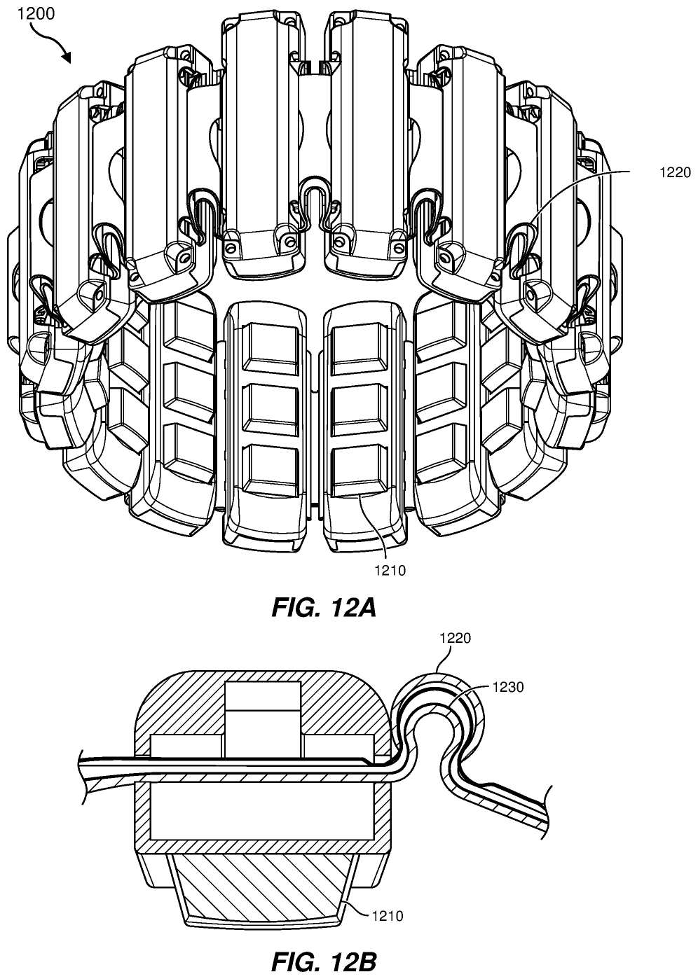

FIG. 12A illustrates an example human-machine interface (also referred to herein as an EMG control interface) configured to be worn around a user's lower arm or wrist as a wearable system 1200. In this example, the wearable system 1200 may include sixteen neuromuscular sensors 1210 (e.g., EMG sensors) arranged circumferentially around an elastic band 1220 with an interior surface 1230 configured to contact a user's skin. However, any suitable number of neuromuscular sensors may be used. The number and arrangement of neuromuscular sensors may depend on the particular application for which the wearable device is used. For example, a wearable armband or wristband can be used to generate control information for controlling an augmented reality system, a robot, controlling a vehicle, scrolling through text, controlling a virtual avatar, or any other suitable control task.

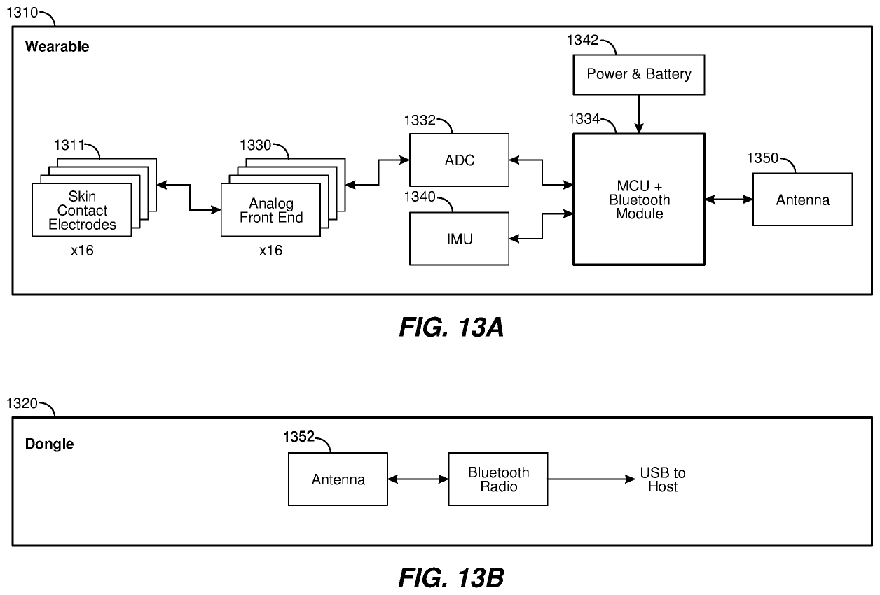

As shown in FIGS. 12A and 12B, the sensors may be coupled together using flexible electronics incorporated into the wireless device. FIG. 12B illustrates a cross-sectional view through one of the sensors of the wearable device shown in FIG. 12A. In some embodiments, the output of one or more of the sensing components can be optionally processed using hardware signal processing circuitry (e.g., to perform amplification, filtering, and/or rectification). In other embodiments, at least some signal processing of the output of the sensing components can be performed in software. Thus, signal processing of signals sampled by the sensors can be performed in hardware, software, or by any suitable combination of hardware and software, as aspects of the technology described herein are not limited in this respect. A non-limiting example of a signal processing chain used to process recorded data from the neuromuscular sensors 1210 is discussed in more detail below with reference to FIGS. 13A and 13B.

FIGS. 13A and 13B illustrate an example schematic diagram with internal components of a wearable system with EMG sensors. As shown, the wearable system may include a wearable portion 1310 (FIG. 13A) and a dongle portion 1320 (FIG. 13B) in communication with the wearable portion 1310 (e.g., via BLUETOOTH or another suitable wireless communication technology). As shown in FIG. 13A, the wearable portion 1310 may include skin contact electrodes 1311, examples of which are described in connection with FIGS. 12A and 12B. The output of the skin contact electrodes 1311 may be provided to analog front end 1330, which may be configured to perform analog processing (e.g., amplification, noise reduction, filtering, etc.) on the recorded signals. The processed analog signals may then be provided to analog-to-digital converter 1332, which may convert the analog signals to digital signals that can be processed by one or more computer processors. An example of a computer processor that may be used in accordance with some embodiments is microcontroller (MCU) 1334, illustrated in FIG. 13A. As shown, MCU 1334 may also include inputs from other sensors (e.g., IMU sensor 1340), and a power and battery module 1342. The output of the processing performed by the MCU 1334 may be provided to an antenna 1350 for transmission to the dongle portion 1320 shown in FIG. 13B.

The dongle portion 1320 may include an antenna 1352, which may be configured to communicate with the antenna 1350 included as part of the wearable portion 1310. Communication between the antennas 1350 and 1352 may occur using any suitable wireless technology and protocol, non-limiting examples of which include radiofrequency signaling and BLUETOOTH. As shown, the signals received by the antenna 1352 of the dongle portion 1320 may be provided to a host computer for further processing, display, and/or for effecting control of a particular physical or virtual object or objects.

Although the examples provided with reference to FIGS. 12A-12B and FIGS. 13A-13B are discussed in the context of interfaces with EMG sensors, the techniques described herein for reducing electromagnetic interference can also be implemented in wearable interfaces with other types of sensors including, but not limited to, mechanomyography (MMG) sensors, sonomyography (SMG) sensors, and electrical impedance tomography (EIT) sensors. The techniques described herein for reducing electromagnetic interference can also be implemented in wearable interfaces that communicate with computer hosts through wires and cables (e.g., USB cables, optical fiber cables, etc.).

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present disclosure includes at least one non-transitory computer readable storage medium (e.g., a computer memory, a portable memory, a compact disk, etc.) encoded with a computer program (e.g., a plurality of instructions), that, when executed on a processor, performs the above-discussed functions of the embodiments of the present disclosure. The computer-readable storage medium can be transportable such that the program stored thereon can be loaded onto any computer resource to implement the aspects of the present disclosure discussed herein. In addition, it should be appreciated that the reference to a computer program that, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present disclosure.

Various aspects of the present disclosure may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the present disclosure may be implemented as one or more methods, of that an example has been provided. The acts performed as part of the method(s) may be ordered in any suitable way. Accordingly, embodiments may be constructed in that acts are performed in an order different than illustrated, that may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in that acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the present disclosure in detail, various modifications and improvements will readily occur to those skilled in the art. Such modifications and improvements are intended to be within the spirit and scope of the present disclosure. Accordingly, the foregoing description is by way of example only, and is not intended as limiting.

By way of non-limiting examples, the following embodiments are included in the present disclosure.

Example 1: A watch, including: a watch band; a watch body that includes a front surface and a rear surface, wherein the rear surface is configured to contact a user's wrist when the watch body is attached to the watch band and donned by the user; at least one light emitting diode on the rear surface of the watch body; and an image sensor on the rear surface of the watch body, wherein: the at least one light emitting diode is configured to provide a light source for a heart rate monitoring function of the watch when the watch body is attached to the watch band; and the at least one light emitting diode is configured to provide a light source for the image sensor when the watch body is detached from the watch band.

Example 2: The watch of Example 1, further including at least one heart rate monitor sensor on the rear surface of the watch body, wherein the at least one heart rate monitor sensor is configured to detect reflected light from the at least one light emitting diode to perform the heart rate monitoring function.

Example 3: The watch of Example 2, wherein the at least one light emitting diode, the image sensor, and the heart rate monitor sensor are individually disposed on a printed circuit board.

Example 4: The watch of Example 2, wherein the at least one light emitting diode, the image sensor, and the heart rate monitor sensor are integrated into a multichip module.

Example 5: The watch of any of Examples 2 through 4, wherein the at least one light emitting diode includes a first light emitting diode configured to function as a flash for the image sensor and a second light emitting diode configured to function as a light source for the heart rate monitor sensor.

Example 6: The watch of any of Examples 1 through 5, wherein the at least one light emitting diode includes a single light emitting diode package.

Example 7: The watch of Example 6, wherein the single light emitting diode package is positioned in a central region of the rear face of the watch body.

Example 8: The watch of any of Examples 1 through 7, wherein the at least one light emitting diode emits light through a single lens.

Example 9: The watch of Example 8, wherein the single lens includes one of: a Fresnel lens or a total internal reflection lens.

Example 10: The watch of any of Examples 1 through 9, further including a coupling mechanism for removably attaching the watch body to the watch band.

Example 11: The watch of any of Examples 1 through 10, wherein the at least one light emitting diode is configured to emit light in multiple different colors.

Example 12: The watch of any of Examples 1 through 11, wherein the at least one light emitting diode is configured to emit visible light and infrared light.

Example 13: A wrist-wearable device, which may include: a body, including: a front face and an opposite rear face, the front face including a display for displaying content to a user; an image sensor positioned in the rear face; a heart rate monitor sensor positioned in the rear face; and a light-emitting diode positioned in the rear face and configured to provide a flash for the image sensor and to provide light for the heart rate monitor sensor; and a band configured to support the body on a wrist of the user.

Example 14: The wrist-wearable device of Example 13, wherein: the light emitting diode is configured to emit infrared light and the heart rate monitor sensor is configured to sense infrared light when the body and band are worn on the wrist of the user; and the light emitting diode is configured to emit visible light and the image sensor is configured to capture an image when the body is not worn on the wrist of the user.

Example 15: The wrist-wearable device of Example 13 or Example 14, wherein the light emitting diode includes a red-green-blue and infrared light emitting diode package.

Example 16: The wrist-wearable device of any of Examples 13 through 15, wherein the body further includes another image sensor positioned in the front face.

Example 17: The wrist-wearable device of any of Examples 13 through 16, wherein the light emitting diode is configured to project light that covers an entire image capture area of the image sensor with a margin around the image capture area of the image sensor.

Example 18: The wrist-wearable device of any of Examples 13 through 17, wherein the body includes a watch band.

Example 19: A non-transitory computer-readable medium that may include one or more computer-executable instructions that, when executed by at least one processor of a computing device, cause the computing device to: determine, with a sensor, that a watch body is attached to a watch band, wherein the watch body includes a front surface and a rear surface that is configured to contact a user's wrist when the watch body is attached to the watch band and donned by the user; when the watch body is attached to the watch band, cause at least one light emitting diode on a rear surface of the watch body to emit light for a heart rate monitoring function of the watch body; determine, with the sensor, that the watch body is detached from the watch band; and when the watch body is detached from the watch band, cause the at least one light emitting diode on the rear surface of the watch body to emit light for an image sensor on the rear surface of the watch body.

Example 20: The non-transitory computer-readable medium of Example 19, wherein causing the at least one light emitting diode to emit the light for the heart rate monitoring function comprises causing the at least one light emitting diode to emit at least infrared light.

Example 21: A wearable device, which may include: a display body including a display in a front face and an image sensor, a heart rate monitor sensor, and at least one light emitting diode in an opposing rear face, wherein the display is configured to display content to a user and the at least one light emitting diode is configured to emit light for illuminating a scene for capturing an image with the image sensor and to emit light for the heart rate monitor sensor; a band shaped and sized for wearing on the user's body; and a coupling mechanism configured for removably coupling the display body to the band.

Example 22: The wearable device of Example 21, wherein the at least one light emitting diode includes a single light emitting diode package configured to emit visible light and infrared light through a common lens.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A watch, comprising:
a watch band;
a watch body that includes a front surface and a rear surface, wherein the rear surface is configured to contact a user's wrist when the watch body is attached to the watch band and donned by the user;
at least one light emitting diode on the rear surface of the watch body; and
an image sensor on the rear surface of the watch body, wherein:
the at least one light emitting diode is configured to provide a light source for a heart rate monitoring function of the watch when the watch body is attached to the watch band; and
the at least one light emitting diode is configured to provide a light source for the image sensor when the watch body is detached from the watch band.

2. The watch of claim 1, further comprising at least one heart rate monitor sensor on the rear surface of the watch body, wherein the at least one heart rate monitor sensor is configured to detect reflected light from the at least one light emitting diode to perform the heart rate monitoring function.

3. The watch of claim 2, wherein the at least one light emitting diode, the image sensor, and the heart rate monitor sensor are individually disposed on a printed circuit board.

4. The watch of claim 2, wherein the at least one light emitting diode and the image sensor are integrated into a rear face of the watch body configured to be adjacent to a user's skin when the watch body is worn by the user.

5. The watch of claim 2, wherein the at least one light emitting diode comprises:
a first light emitting diode configured to function as a flash for the image sensor; and
a second light emitting diode configured to function as a light source for the heart rate monitor sensor.

6. The watch of claim 1, wherein the at least one light emitting diode comprises a single light emitting diode package.

7. The watch of claim 6, wherein the single light emitting diode package is positioned in a central region of the rear face of the watch body.

8. The watch of claim 1, wherein the at least one light emitting diode emits light through a single lens.

9. The watch of claim 8, wherein the single lens comprises one of: a Fresnel lens or a total internal reflection lens.

10. The watch of claim 1, further comprising a coupling mechanism for removably attaching the watch body to the watch band.

11. The watch of claim 1, wherein the at least one light emitting diode is configured to emit light in multiple different colors.

12. The watch of claim 1, wherein the at least one light emitting diode is configured to emit visible light and infrared light.

13. A wrist-wearable device, comprising:
a body, comprising:
a front face and an opposite rear face, the front face including a display for displaying content to a user;
an image sensor positioned in the rear face;
a heart rate monitor sensor positioned in the rear face; and
a light emitting diode positioned in the rear face and configured to provide a flash for the image sensor and to provide light for the heart rate monitor sensor; and
a band configured to support the body on a wrist of the user.

14. The wrist-wearable device of claim 13, wherein:
the light emitting diode is configured to emit infrared light and the heart rate monitor sensor is configured to sense infrared light when the body and band are worn on the wrist of the user; and
the light emitting diode is configured to emit visible light and the image sensor is configured to capture an image when the body is not worn on the wrist of the user.

15. The wrist-wearable device of claim 13, wherein the light emitting diode comprises a red-green-blue and infrared light emitting diode package.

16. The wrist-wearable device of claim 13, wherein the body further comprises another image sensor positioned in the front face.

17. The wrist-wearable device of claim 13, wherein the light emitting diode is configured to project light that covers an entire image capture area of the image sensor with a margin around the image capture area of the image sensor.

18. The wrist-wearable device of claim 13, wherein the body comprises a watch body and the band comprises a watch band.

19. A non-transitory computer-readable medium comprising one or more computer-executable instructions that, when executed by at least one processor of a computing device, cause the computing device to:
determine, with a sensor, that a watch body is attached to a watch band, wherein the watch body includes a front surface and a rear surface that is configured to contact a user's wrist when the watch body is attached to the watch band and donned by the user;
when the watch body is attached to the watch band, cause at least one light emitting diode on a rear surface of the watch body to emit light for a heart rate monitoring function of the watch body;
determine, with the sensor, that the watch body is detached from the watch band; and
when the watch body is detached from the watch band, cause the at least one light emitting diode on the rear surface of the watch body to emit light for an image sensor on the rear surface of the watch body.

20. The non-transitory computer-readable medium of claim 19, wherein causing the at least one light emitting diode to emit the light for the heart rate monitoring function comprises causing the at least one light emitting diode to emit at least infrared light.
